(19) United States
(12) Patent Application Publication
(10) Pub. No.: US 2024/0408347 A1
(43) Pub. Date: Dec. 12, 2024

(54) ERGONOMIC PILLOW SYSTEM, PILLOW INSERT APPARATUS, PILLOW COVER SYSTEM, AND METHOD OF USE

(71) Applicant: Casey D'Arcy, Bristow, VA (US)

(72) Inventor: Casey D'Arcy, Bristow, VA (US)

(21) Appl. No.: 18/739,410

(22) Filed: Jun. 11, 2024

Related U.S. Application Data
(60) Provisional application No. 63/472,577, filed on Jun. 12, 2023.

(51) Int. Cl. A61M 21/02; A47G 9/10; A61M 21/00

(52) U.S. Cl. CPC A61M 21/02; A47G 9/1081; A61M 2021/0022; A61M 2205/3303; A61M 2205/3331; A61M 2205/50

(57) ABSTRACT

An ergonomic pillow system, a pillow insert apparatus, a pillow cover system, and methods of use are presented. An ergonomic pillow system, a pillow insert apparatus, a pillow cover system, and methods of use for sleeping are presented. Said another way, the following provides a unique ergonomic pillow which provides benefits of comfort and alignment for a user via a contoured insert. The system is made comfortable through the methods of use and the layers further described.

### CROSS-REFERENCE TO RELATED APPLICATIONS

The present application claims priority to the U.S. Provisional Patent Application No. 63/472,577 which was filed on Jun. 12, 2023, which is hereby incorporated by reference herein in its entirety, including any figures, tables, or drawings.

### FIELD OF THE INVENTION

This disclosure relates to an ergonomic pillow system, a pillow insert apparatus, a pillow cover system, and methods of use. More specifically, and without limitation, the present disclosure relates to an ergonomic pillow for use while sleeping. More specifically, and without limitation, the present disclosure relates to a pillow cover and a contoured insert. More specifically, and without limitation, the present disclosure relates to contoured insert for ergonomic purposes encased in a comfortable material.

### BACKGROUND OF THE INVENTION

Sleeping and relaxing are universal across all cultures. Sleeping, in many estimates, is stated to take up about one-third of human lives on the planet. Despite this common and regularity of the engaged activity, few developments have advanced the art of sleeping. While sleeping has been advanced greatly on the mattress front and through various comfort methods, the ergonomics of sleeping and particularly the ergonomics of the shoulders, neck, and head continue to be a struggle.

Pillows are well known in the art. Pillows provide comfort and are known generally for use during sleep, relaxation, therapy, and even decoration. Pillows are used in various ways and by various species. Various pillow types include throw pillows, body pillows, decorative pillows and others. Sleep pillows and other types are important for positioning oneself in a comfortable position. The position of comfort is particularly important during sleep, for which the user will likely spend a long time in a particular position.

Pillows that help make comfortable the act of sleeping are a form of bedding that supports the head and neck, generally. Other types of pillows are designed to support the body when lying down or sitting. There are also pillows that consider human body shape for increased comfort during sleep. Decorative pillows used on beds, couches or chairs are sometimes referred to as cushions. Thus, a pillow which is ergonomically supportive and comfortable, both, during sleep is desirable and important to the health of a user.

Generally speaking, a conventional commercial pillow comprises a single material, often cotton or feathers and/or feather encased in cotton. Users often choose a pillow with an appropriate height and rigidity, or stiffness, in order to position the body for increased comfort and ability to sleep. Thus, a good pillow causes a user to relax and make the cervical spine, shoulders, neck, and head comfortable.

One key consideration known in the art is the use of technology. Typing on a keyboard, engaging with computers, smartphones, and the like are known to change positioning of the body for long periods of time. As technology use increases the spinal health of a user decreases. Many spend most of their waking hours looking down at computers and/or smart phones. The strain put on the muscles, ligaments, discs, and spinal nerves of users of technology is great. Thus, there is a need in the art for a pillow system which provides rest, relaxation, and/or sleep in proper spinal alignment; especially an alignment which counteracts the rigors of daily uses of technology.

Thus, there is a long-felt need in the art for an ergonomic pillow system, a pillow insert apparatus, a pillow cover system, and methods of use that improve upon the state of the art.

The disclosure herein provides these advantages and others as will become clear from the specification and claims provided.

### SUMMARY OF THE INVENTION

Thus, the present disclosure provides an ergonomic pillow solution which is comfortable while also providing proper spine alignment during sleep; along with other ergonomic benefits such as combating positions during technology use and the causes of those positions on the human body. In other words, an ergonomic pillow system, a pillow insert apparatus, a pillow cover system, and methods of use are presented which improve the mechanics of the human body when used, for a variety of user types.

Said another way, the present disclosure provides the state of the art with an ergonomic pillow system, a pillow insert apparatus, a pillow cover system, and methods of use. More specifically, and without limitation, the present disclosure relates to an ergonomic pillow for use while sleeping. More specifically, and without limitation, the present disclosure relates to a pillow cover and a contoured insert. More specifically, and without limitation, the present disclosure relates to contoured insert for ergonomic purposes encased in a comfortable material.

Thus, it is a primary object of the disclosure to provide an ergonomic pillow system, a pillow insert apparatus, a pillow cover system, and methods of use that improve upon the state of the art.

Another object of the disclosure is to provide an ergonomic pillow system, a pillow insert apparatus, a pillow cover system, and methods of use that provides a contoured pillow insert.

Yet another object of the disclosure is to provide an ergonomic pillow system, a pillow insert apparatus, a pillow cover system, and methods of use that provides a plurality of ovular and/or circular concavities with varying depths and shapes.

Another object of the disclosure is to provide an ergonomic pillow system, a pillow insert apparatus, a pillow cover system, and methods of use that provide appropriate anatomical sleep position.

Yet another object of the disclosure is to provide an ergonomic pillow system, a pillow insert apparatus, a pillow cover system, and methods of use that maintains ergonomic sleep position for the duration of the sleeping process.

Another object of the disclosure is to provide an ergonomic pillow system, a pillow insert apparatus, a pillow cover system, and methods of use that provides for an aligned spine.

Yet another object of the disclosure is to provide an ergonomic pillow system, a pillow insert apparatus, a pillow cover system, and methods of use that provide a neutral spine.

Another object of the disclosure is to provide an ergonomic pillow system, a pillow insert apparatus, a pillow cover system, and methods of use that provides for improved health.

Yet another object of the disclosure is to provide an ergonomic pillow system, a pillow insert apparatus, a pillow cover system, and methods of use that provide improved and reduced strain on muscles and ligaments.

Another object of the disclosure is to provide an ergonomic pillow system, a pillow insert apparatus, a pillow cover system, and methods of use that reduces strain on discs.

Yet another object of the disclosure is to provide an ergonomic pillow system, a pillow insert apparatus, a pillow cover system, and methods of use that reduces strain on the spine.

Another object of the disclosure is to provide an ergonomic pillow system, a pillow insert apparatus, a pillow cover system, and methods of use that increase spinal health.

Yet another object of the disclosure is to provide an ergonomic pillow system, a pillow insert apparatus, a pillow cover system, and methods of use that provide spine alignment.

Another object of the disclosure is to provide an ergonomic pillow system, a pillow insert apparatus, a pillow cover system, and methods of use that prevents unwanted positioning of the head and/or neck.

Yet another object of the disclosure is to provide an ergonomic pillow system, a pillow insert apparatus, a pillow cover system, and methods of use that prevents undesired chin to chest flexion.

Another object of the disclosure is to provide an ergonomic pillow system, a pillow insert apparatus, a pillow cover system, and methods of use that prevents undesired ear to shoulder flexion.

Yet another object of the disclosure is to provide an ergonomic pillow system, a pillow insert apparatus, a pillow cover system, and methods of use that improves breathing and combats technology induced strains on the body; and that prevents hyper-kyphosis or rounded shoulders that compresses the lung cavity.

Another object of the disclosure is to provide an ergonomic pillow system, a pillow insert apparatus, a pillow cover system, and methods of use that are high quality.

Yet another object of the disclosure is to provide an ergonomic pillow system, a pillow insert apparatus, a pillow cover system, and methods of use that maintain positional integrity over many uses.

Another object of the disclosure is to provide an ergonomic pillow system, a pillow insert apparatus, a pillow cover system, and methods of use that maintain positional integrity for prolonged periods of time.

Yet another object of the disclosure is to provide an ergonomic pillow system, a pillow insert apparatus, a pillow cover system, and methods of use that are robust.

Another object of the disclosure is to provide an ergonomic pillow system, a pillow insert apparatus, a pillow cover system, and methods of use that provides for both side and back sleeping options.

Yet another object of the disclosure is to provide an ergonomic pillow system, a pillow insert apparatus, a pillow cover system, and methods of use that allows a user to ergonomically sleep on either desired side.

These and other objects, features, or advantages of the present disclosure will become apparent from the specification and claims.

### BRIEF DESCRIPTION OF DRAWINGS

The drawings accompanying and forming part of this specification are included to depict certain aspects of the disclosure.

Fig. 1 is a top, perspective view of the ergonomic pillow system, the view showing an outer cover with an access port in the closed position.

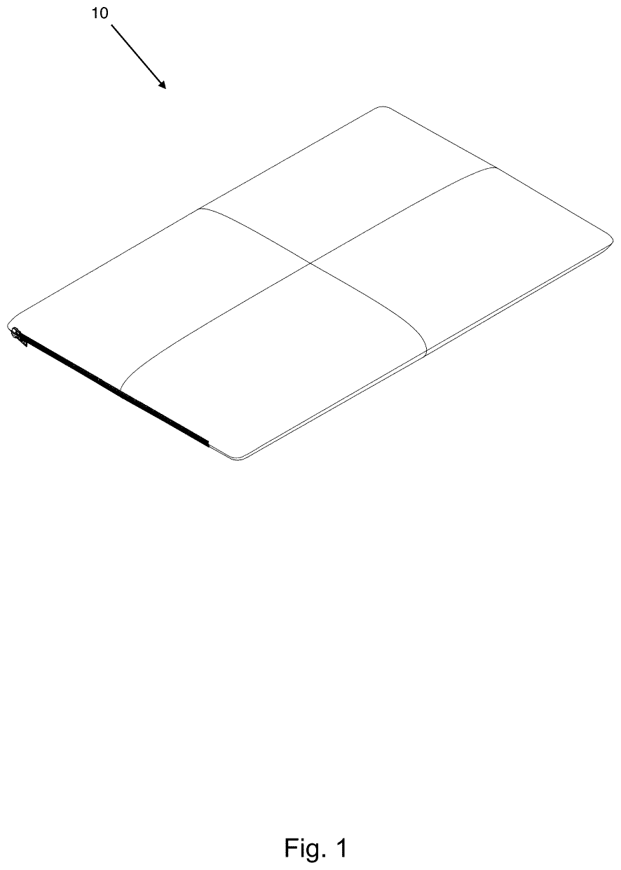

Fig. 2 is a top, perspective view of one embodiment of the ergonomic pillow system; the view showing an outer cover; the view showing an inner cover; the view showing a core.

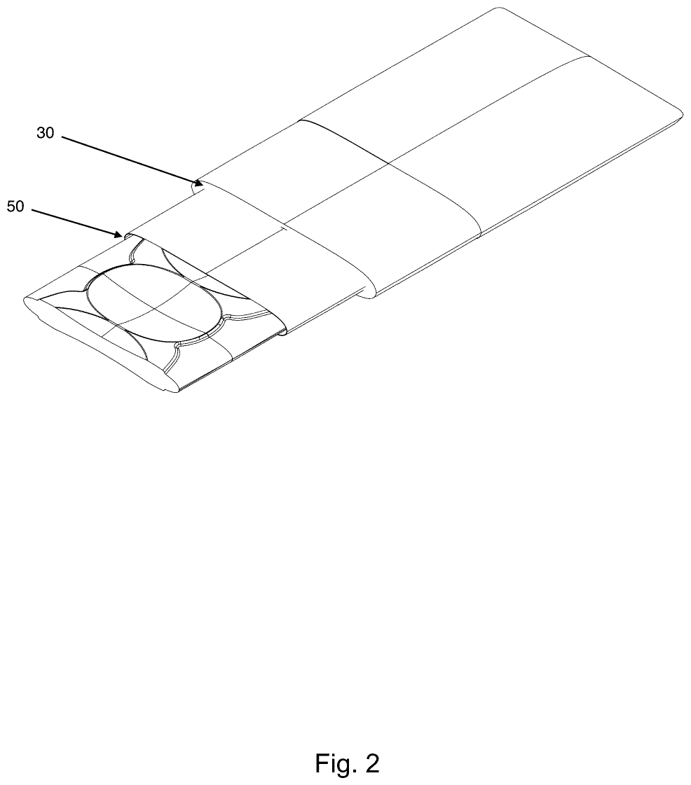

Fig. 3 is a top, perspective view of one embodiment of the ergonomic pillow system; the view showing an outer cover; the view showing an inner cover; the view showing a second inner cover; the view showing a core.

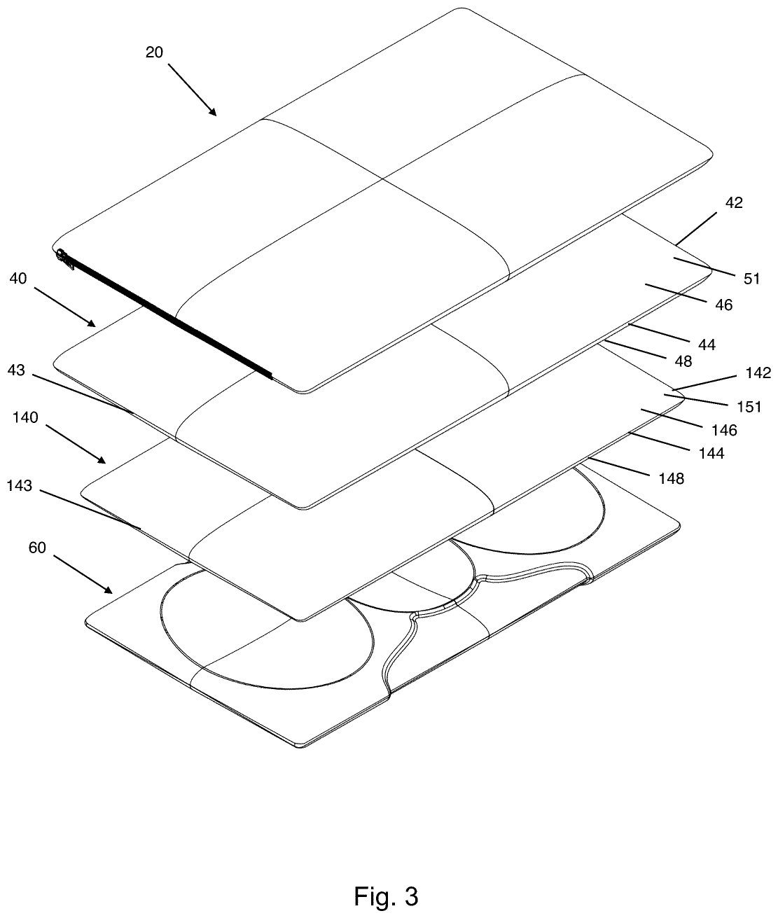

Fig. 4 is a top, perspective view of one embodiment of the ergonomic pillow system; the view showing an outer cover; the view showing an inner cover; the view showing a second inner cover; the view showing a core.

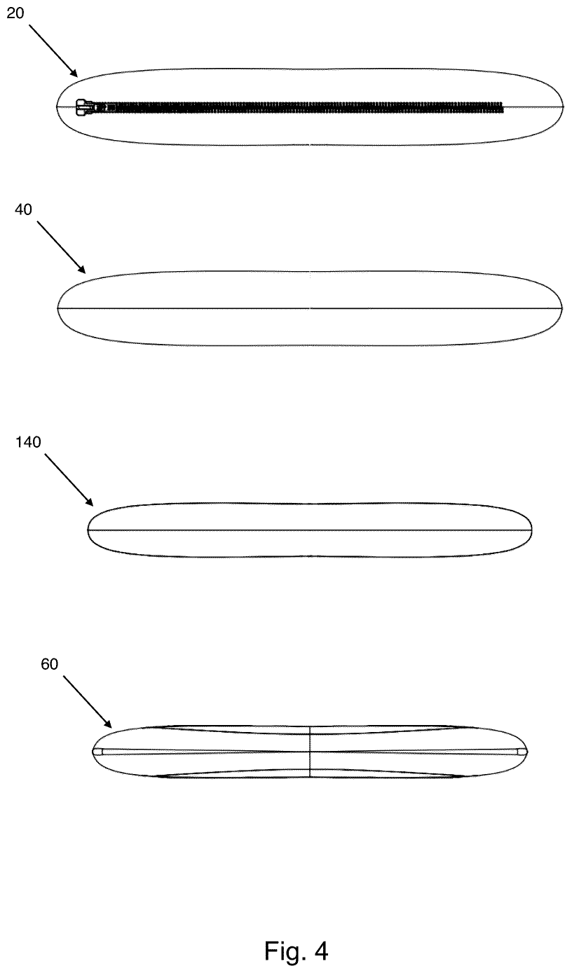

Fig. 5 is a front view of an outer cover; the view also showing a side cross-sectional view of the ergonomic pillow system with a core inside the outer cover; the view also showing a close up view of the cross sectional view.

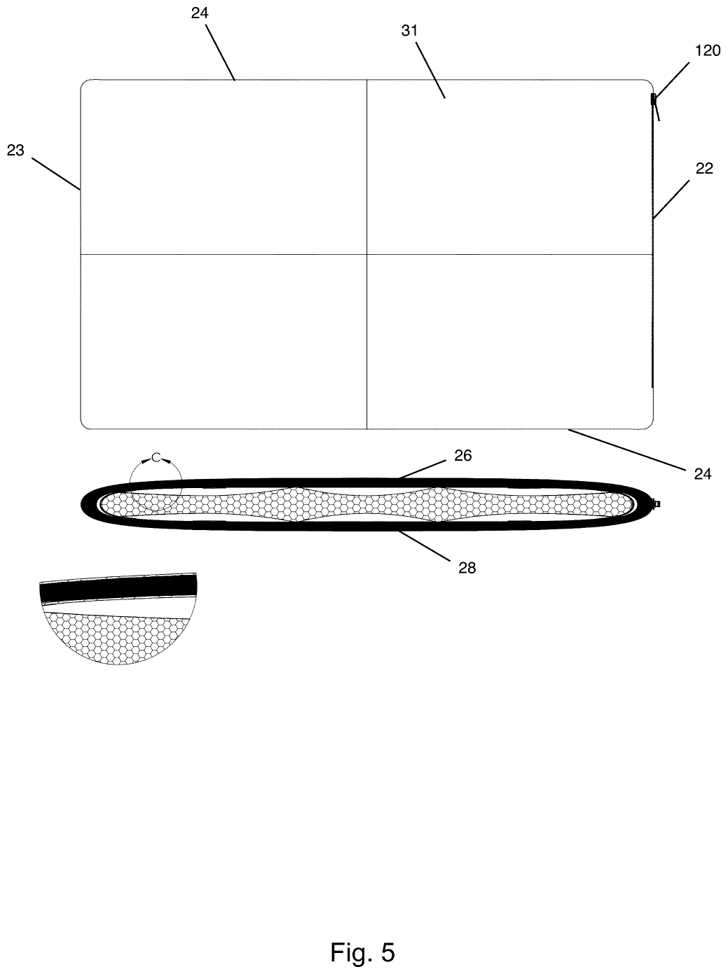

Fig. 6 is a side view of the outer cover; the view also showing a side cross-sectional view of the ergonomic pillow system with a core inside the outer cover.

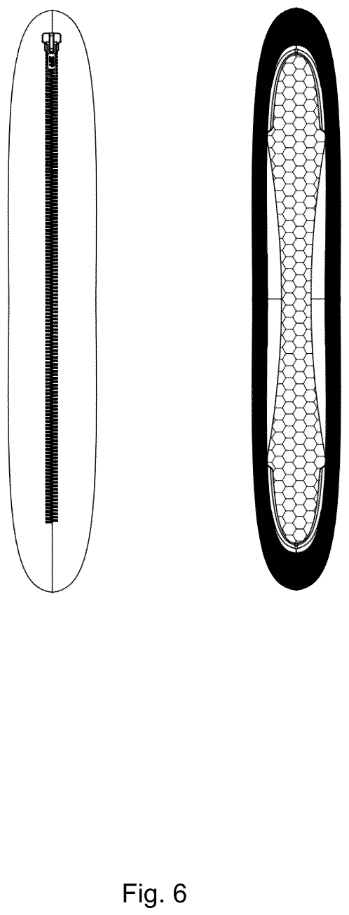

Fig. 7 is a front view of one embodiment of the ergonomic pillow system; the view showing a core; the view showing a cross-sectional view of the core; the cross-sectional view showing interior structure of the core.

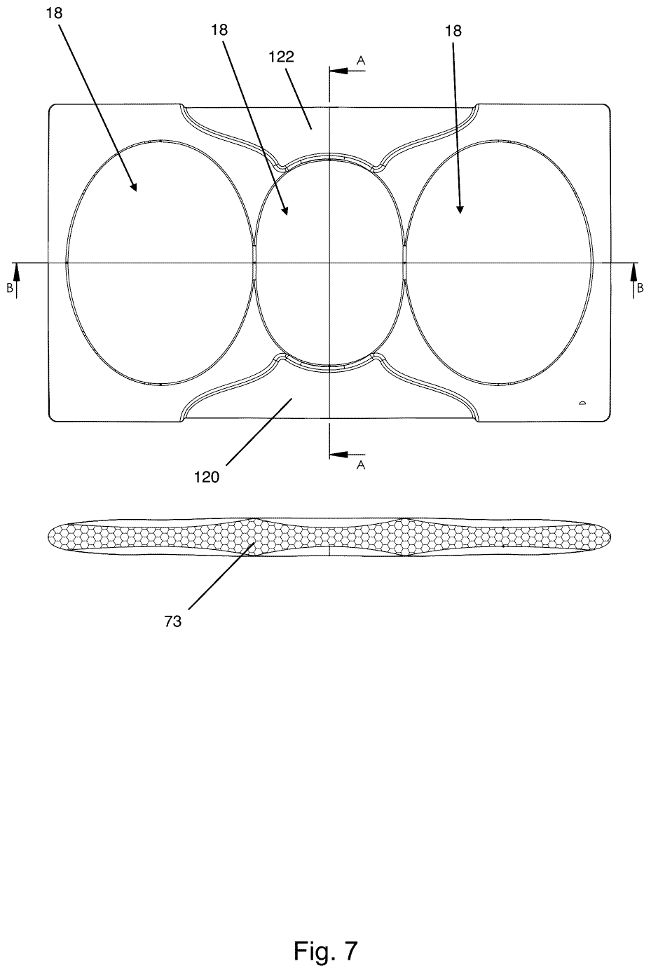

Fig. 8 is a side view of the outer cover; the view also showing a side cross-sectional view of the ergonomic pillow system with a core inside the outer cover.

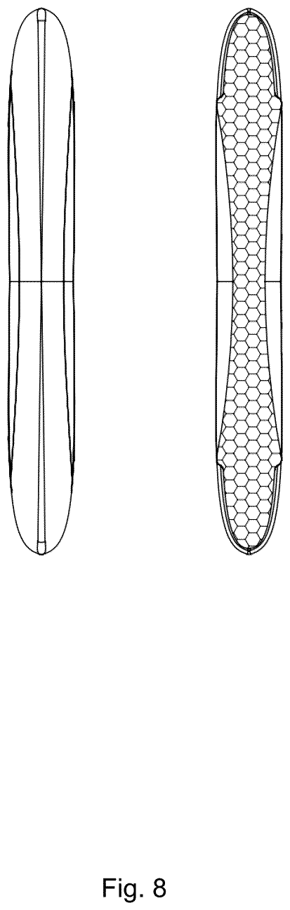

Fig. 9 is a front view of one embodiment of the ergonomic pillow system; the view showing a core; the view showing a cross-sectional view of the core; the cross-sectional view showing interior structure of the core.

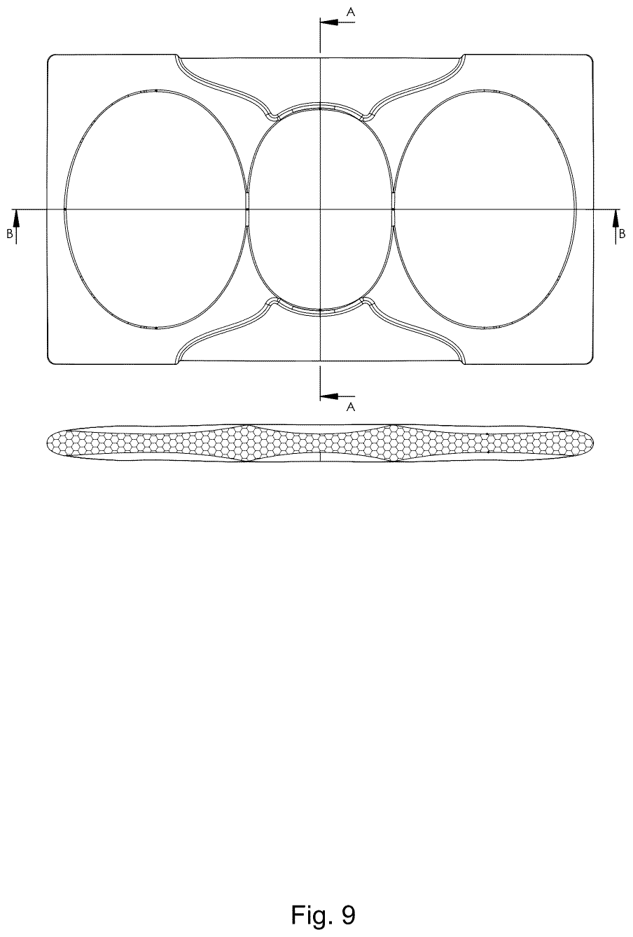

Fig. 10 is a side view of the outer cover; the view also showing a side cross-sectional view of the ergonomic pillow system with a core inside the outer cover.

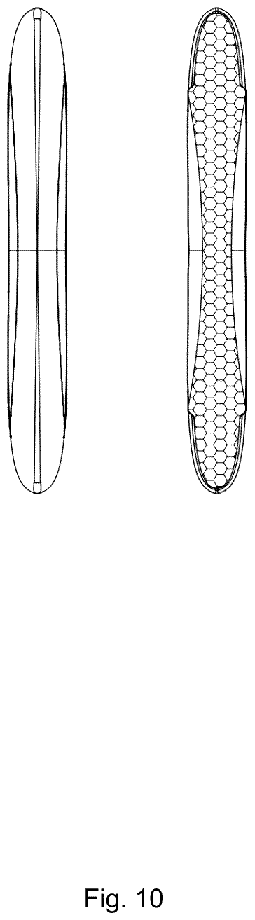

Fig. 11 is a front view of one embodiment of the ergonomic pillow system; the view showing a core; the view showing a cross-sectional view of the core; the cross-sectional view showing interior structure of the core.

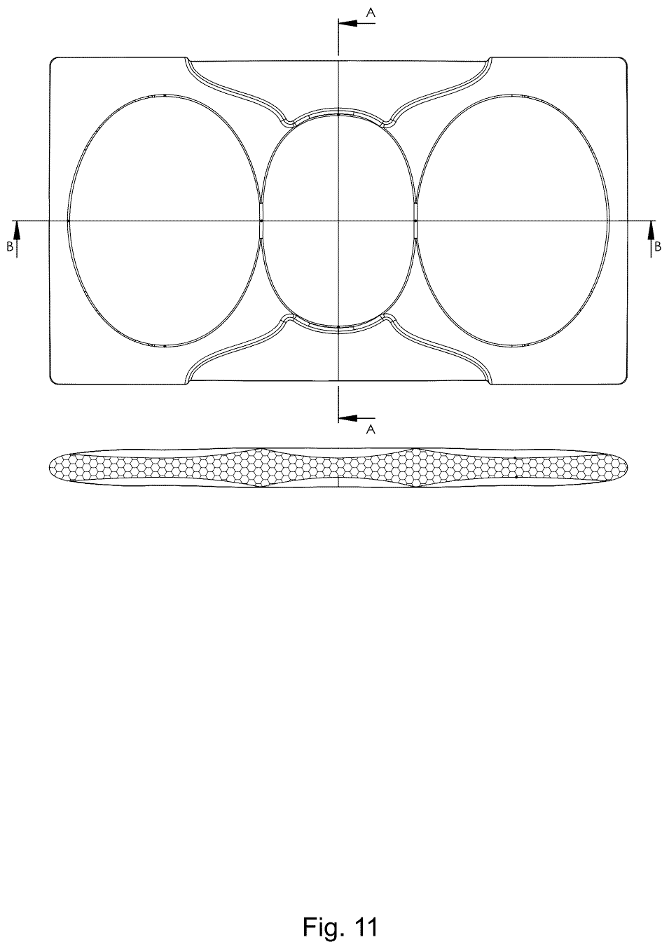

Fig. 12 is a side view of the core of one embodiment of the ergonomic pillow; the view showing a side cross-sectional view of the ergonomic pillow system with a core inside the outer cover.

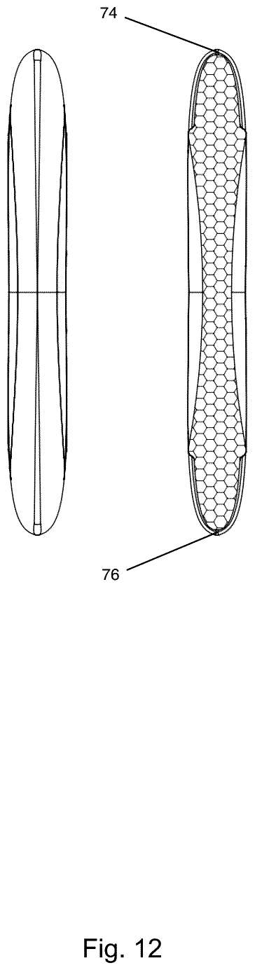

Fig. 13 is a front view of a covering of the system.

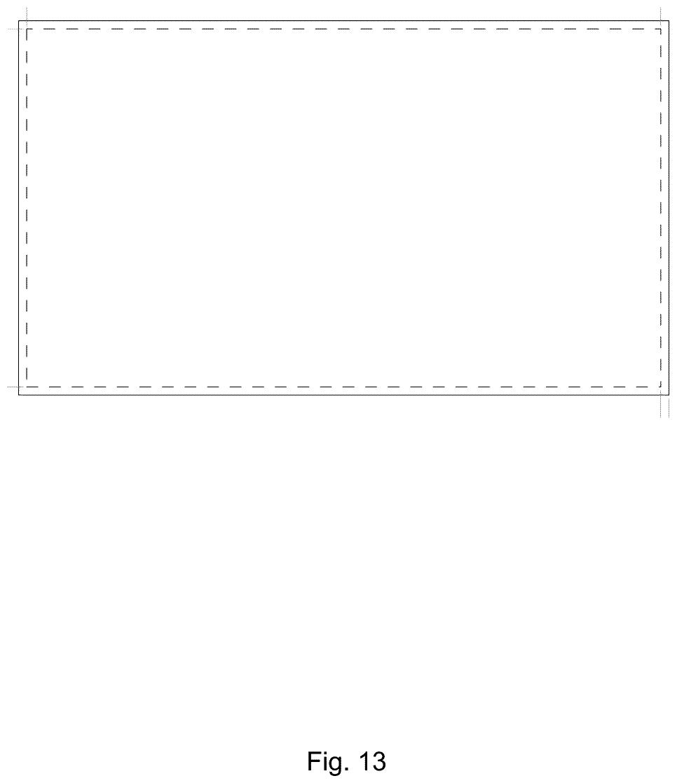

Fig. 14 is a top, perspective view of the core; the view showing one embodiment of the ergonomic pillow system; the view showing a first cavity and a second cavity; the view showing various support and engagement features.

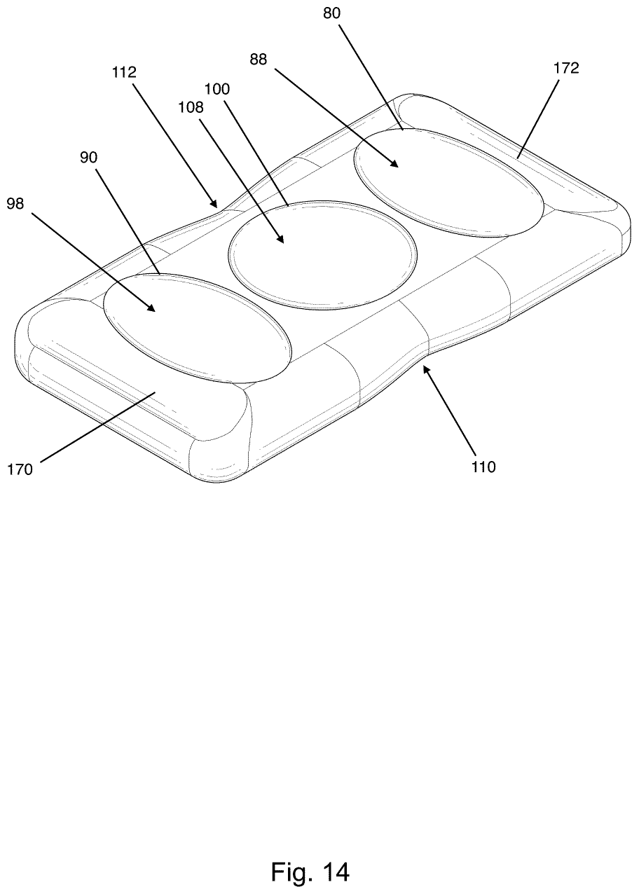

Fig. 15 is a bottom view of the core; the view showing one embodiment of the ergonomic pillow system; the view showing a first cavity and a second cavity; the view showing various support and engagement features.

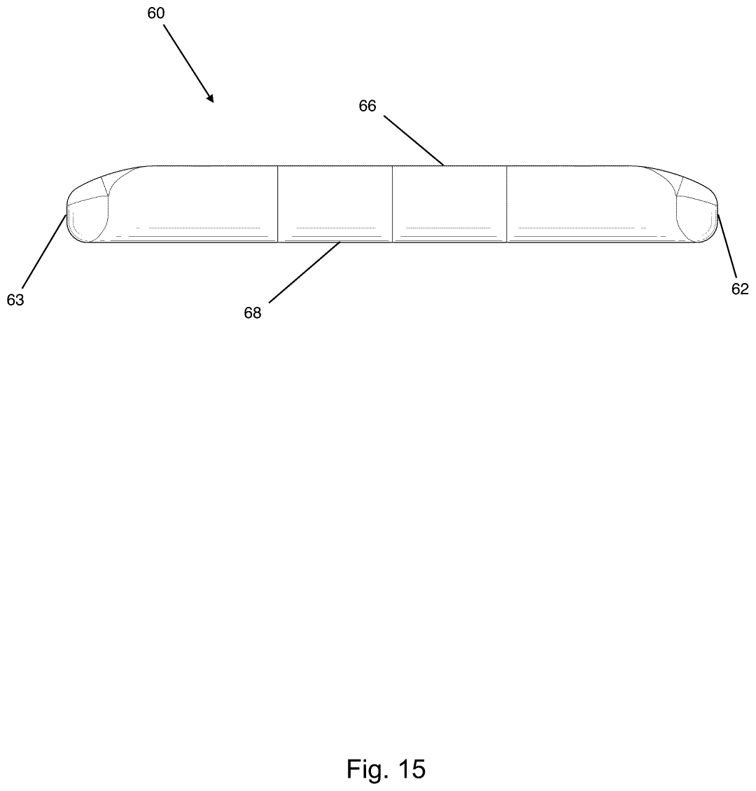

Fig. 16 is a top view of the core; the view showing one embodiment of the ergonomic pillow system; the view showing a first cavity and a second cavity; the view showing various support and engagement features.

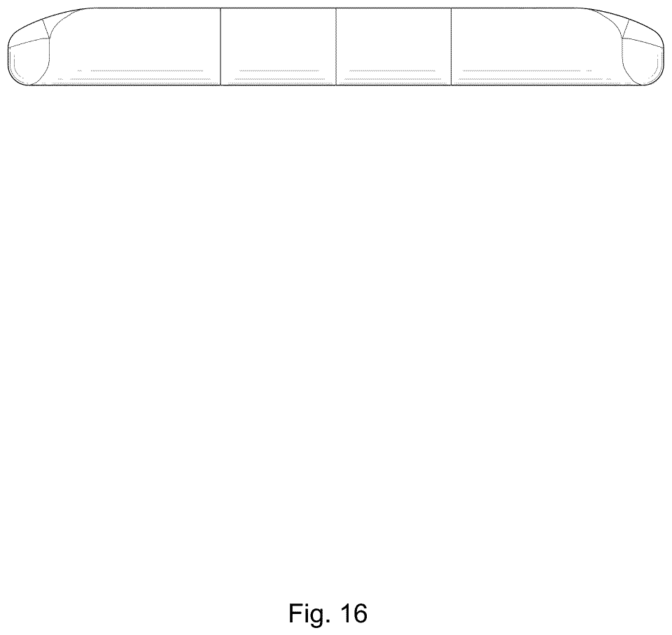

Fig. 17 is a front view of the core; the view showing one embodiment of the ergonomic pillow system; the view showing a first cavity and a second cavity; the view showing various support and engagement features.

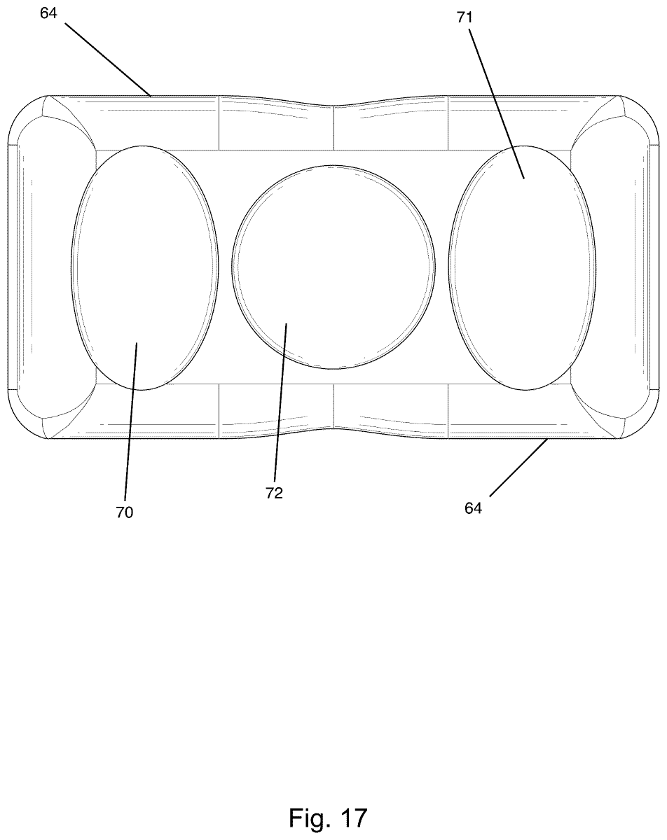

Fig. 18 is a rear view of the core; the view showing one embodiment of the ergonomic pillow system; the view showing a first cavity and a second cavity; the view showing various support and engagement features.

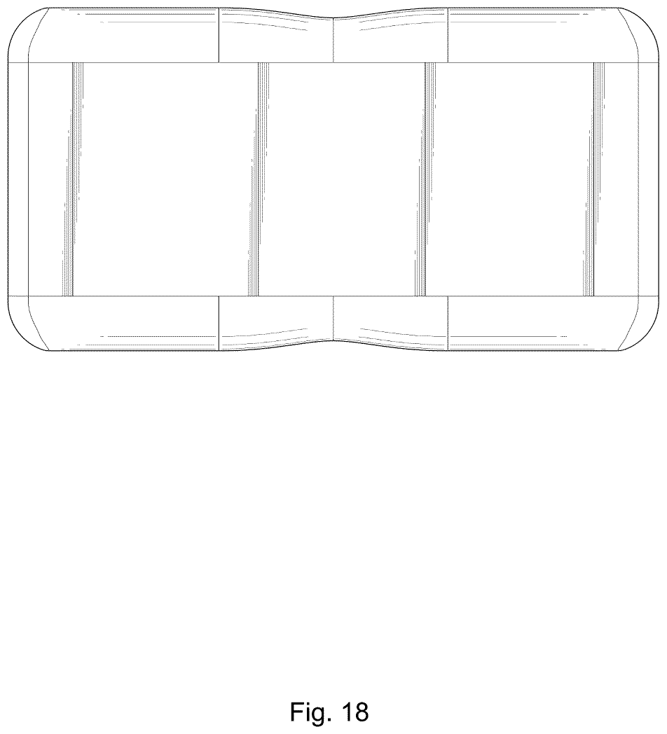

Fig. 19 is a side view of the core; the view showing one embodiment of the ergonomic pillow system; the view showing a first cavity and a second cavity; the view showing various support and engagement features.

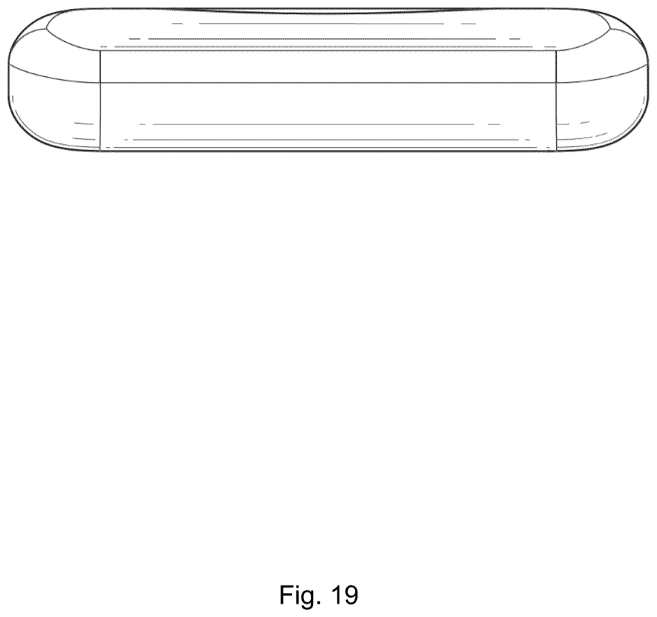

Fig. 20 is a side view of the core; the view showing one embodiment of the ergonomic pillow system; the view showing a first cavity and a second cavity; the view showing various support and engagement features.

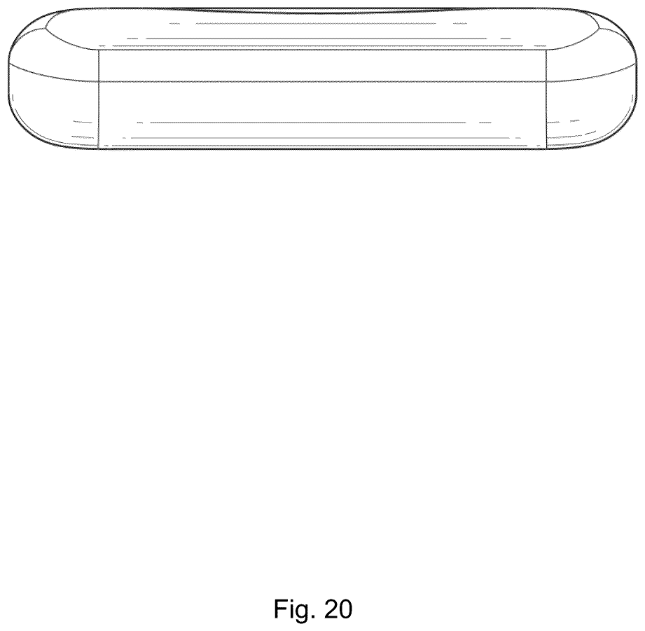

Fig. 21 is a bottom, perspective view of the core; the view showing one embodiment of the ergonomic pillow system; the view showing a first cavity and a second cavity; the view showing various support and engagement features.

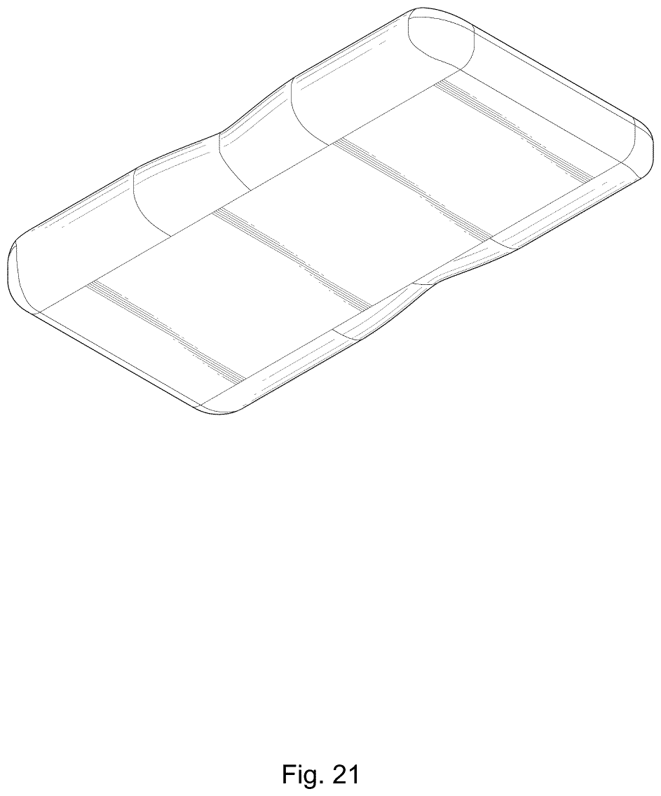

Fig. 22 is a top, perspective view of the core; the view showing one embodiment of the ergonomic pillow system; the view showing a first cavity and a second cavity; the view showing various support and engagement features.

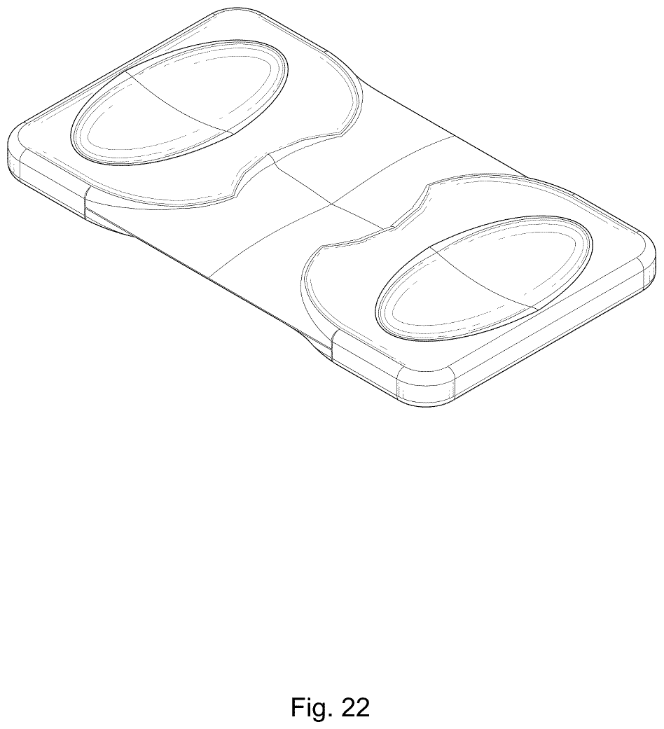

Fig. 23 is a bottom view of the core; the view showing one embodiment of the ergonomic pillow system; the view showing a first cavity and a second cavity; the view showing various support and engagement features.

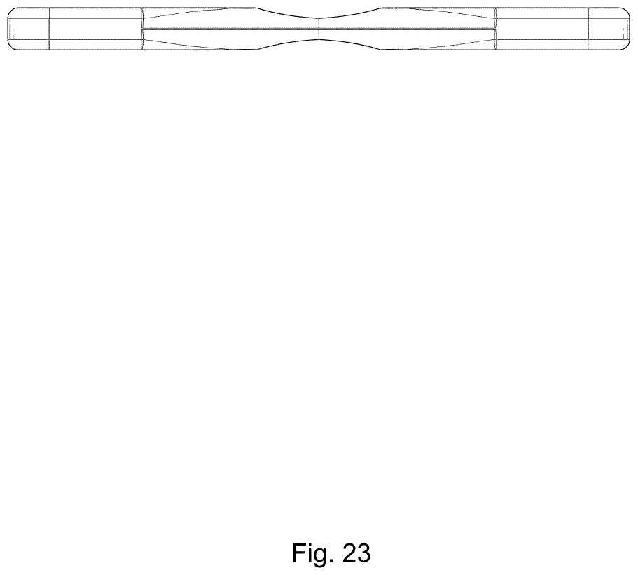

Fig. 24 is a top view of the core; the view showing one embodiment of the ergonomic pillow system; the view showing a first cavity and a second cavity; the view showing various support and engagement features.

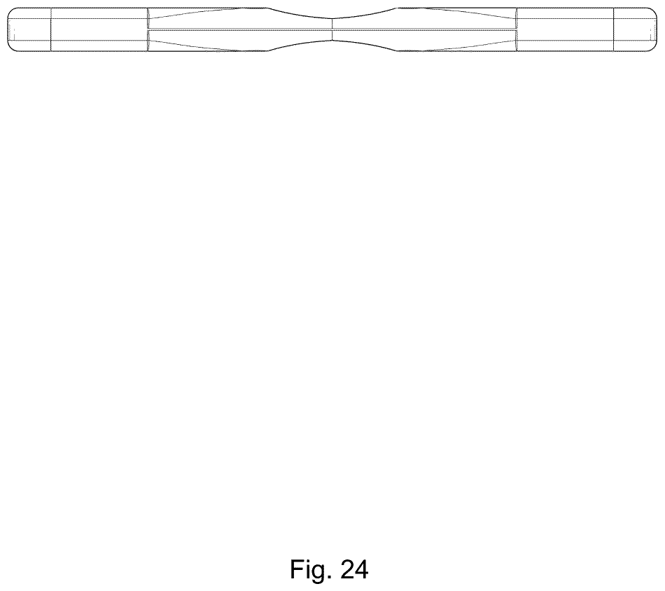

Fig. 25 is a front view of the core; the view showing one embodiment of the ergonomic pillow system; the view showing a first cavity and a second cavity; the view showing various support and engagement features.

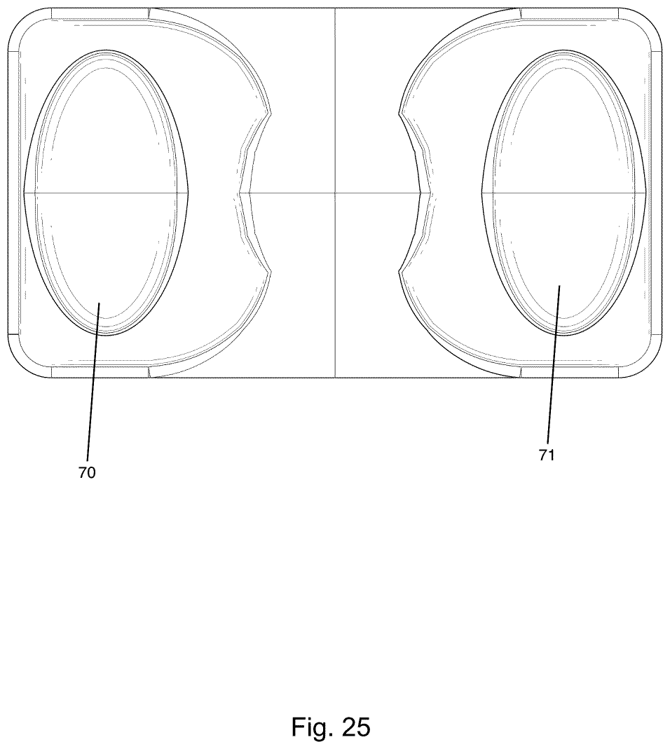

Fig. 26 is a rear view of a double sided embodiment of the core; the view showing one embodiment of the ergonomic pillow system; the view showing a first cavity and a second cavity; the view showing various support and engagement features.

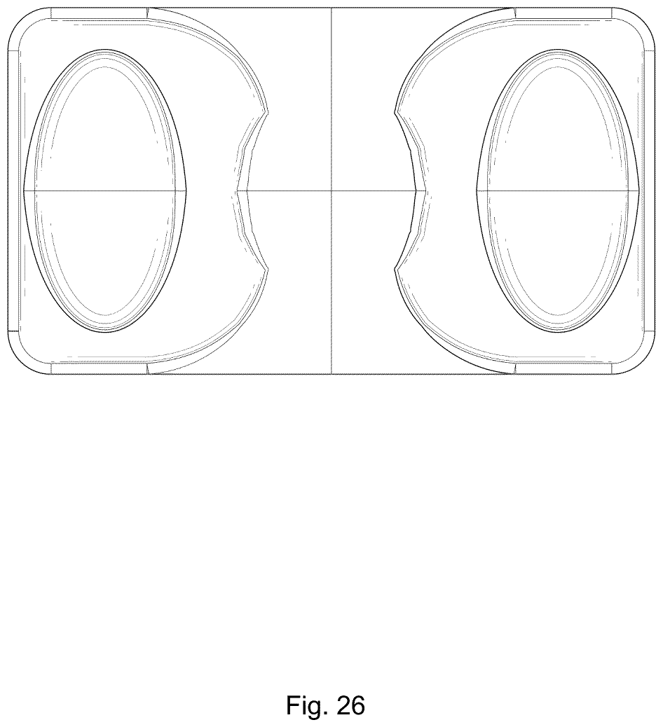

Fig. 27 is a bottom view of a double sided embodiment of the core; the view showing one embodiment of the ergonomic pillow system; the view showing a first cavity and a second cavity; the view showing various support and engagement features.

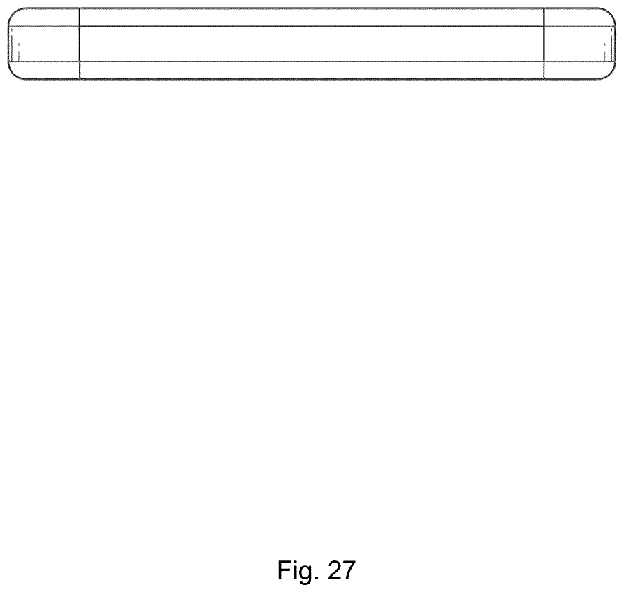

Fig. 28 is a top view of a double sided embodiment of the core; the view showing one embodiment of the ergonomic pillow system; the view showing a first cavity and a second cavity; the view showing various support and engagement features.

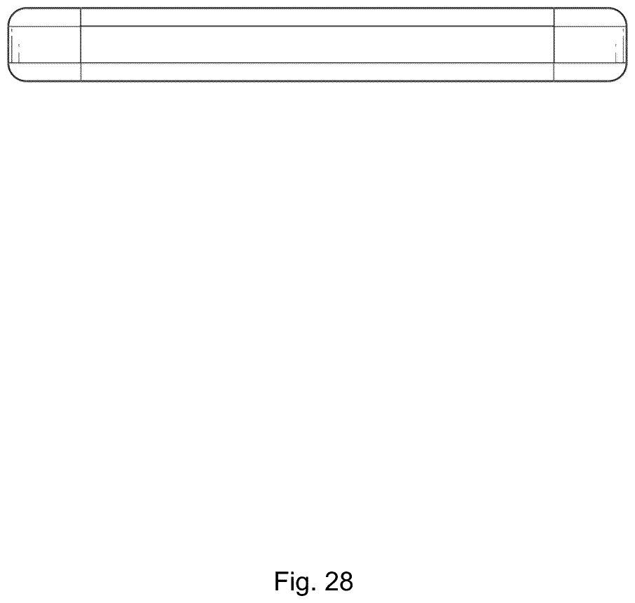

Fig. 29 is a bottom, perspective view of a double sided embodiment of the core; the view showing one embodiment of the ergonomic pillow system; the view showing a first cavity and a second cavity; the view showing various support and engagement features.

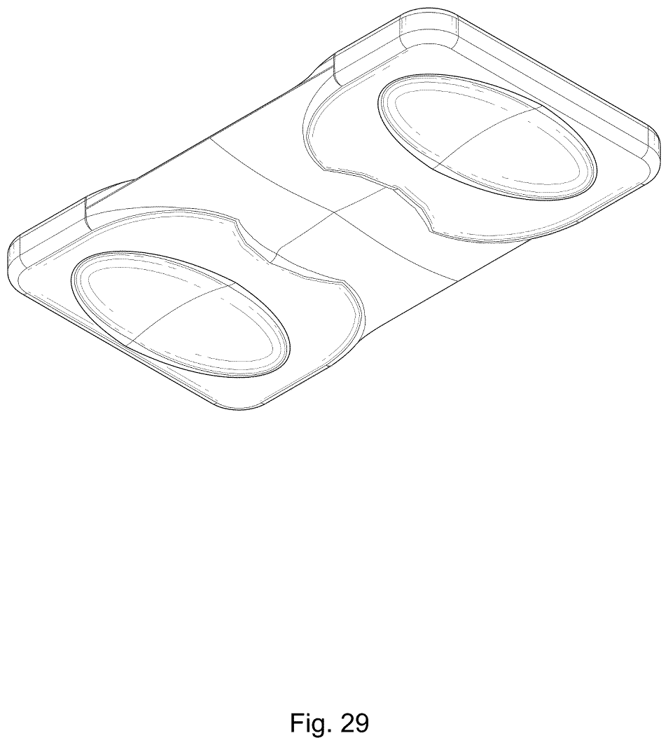

### DESCRIPTION

In the following detailed description, reference is made to the accompanying drawings which form a part hereof, and in which is shown by way of illustration specific embodiments in which the disclosure may be practiced. These embodiments are described in sufficient detail to enable those skilled in the art to practice the disclosure, and it is to be understood that other embodiments may be utilized and that mechanical, procedural, and other changes may be made without departing from the spirit and scope of the disclosure(s). The following detailed description is, therefore, not to be taken in a limiting sense, and the scope of the disclosure(s) is defined only by the appended claims, along with the full scope of equivalents to which such claims are entitled.

As used herein, the terminology such as vertical, horizontal, top, bottom, front, back, end, sides and the like are referenced according to the views, pieces and figures presented. It should be understood, however, that the terms are used only for purposes of description, and are not intended to be used as limitations. Accordingly, orientation of an object or a combination of objects may change without departing from the scope of the disclosure.

Reference throughout this specification to “one embodiment,” “an embodiment,” “one example,” or “an example” means that a particular feature, structure, or characteristic described in connection with the embodiment or example is included in at least one embodiment of the present disclosure. Thus, the appearance of the phrases “in one embodiment,” “in an embodiment,” “one example,” or “an example” in various places throughout this specification are not necessarily all referring to the same embodiment or example. Furthermore, the particular features, structures, databases, or characteristics may be combined in any suitable combinations and/or sub-combinations in one or more embodiments or examples. In addition, it should be appreciated that the figures provided herewith are for explanation purposes to persons ordinarily skilled in the art and that the drawings are not necessarily drawn to scale.

### System

With reference to the figures, an ergonomic pillow system, a pillow insert apparatus, a pillow cover system, and methods of use 10 are presented. Ergonomic pillow system, a pillow insert apparatus, a pillow cover system, and methods of use 10 (hereafter referred to as “ergonomic pillow system”, “pillow apparatus”, “pillow insert device”, “pillow system”, “insert device”, or simply “system”) is formed of any suitable size, shape and design.

In the arrangement shown, as one example, system 10 is configured to provide improved sleep, improved ergonomics, and improved health and well-being of a user. In the arrangement shown, as one example, system 10 provides improved ergonomic position of a user. Furthermore, system 10 maintains improved ergonomics and structure of a sleeper while maintaining comfort throughout sleep and/or the duration of sleep of a user. Furthermore, this improved ergonomic position is very important as about one-third of most user's lives are spent sleeping. Additionally, improved uses of modern technology have caused and continue to cause poor posture and ergonomics which are continued into sleep with devices existing in the art. The present disclosure provides novel solution and prevention of these ailments and issues plaguing the art. Furthermore, the present disclosure prevents unnecessary strain on muscles, ligaments, discs, and nerves which currently plague the state of the art. Furthermore, system 10 is configured to provide spinal alignment during sleep.

In the arrangement shown, as one example, system 10 is configured to provide a unique insert device with a plurality of concavities. The plurality of concavities 18 having varying depths and distinct ovular and/or circular shapes.

In the arrangement shown, as one example, the plurality of concavities are configured to provide improved comfort and ergonomics for a user. Said another way, the center, circular concavity or the second concavity 40 is designed to cradle the head of a user. Cradle and indent may be used synonymously throughout. In the arrangement shown, as one example, the center concavity provides an arch, at the superior and inferior borders, which leads to the pillows edge. This arch feature provides support for the cervical spine of a user during the sleep and/or relaxation period.

Said another way, when a user sleeps, muscle support for the head and/or cranium is relaxed and/or rested. Thus, only the pillow or no device at all supports the weight of the head. In the arrangement shown, as one example, system 10 provides support of the head and/or neck and/or prevents strain on these muscles, ligaments, joints, and the like; while providing alignment. Said another way, the center concavity and/or second concavity support the head. System 10 provides varying thickness which bring the head to a neutral state and prevent the head from falling to the side. Furthermore, the center concavity also provides that the head can still be close to the mattress while providing these features while maintaining the head over the shoulders with spine alignment.

Said another way, and in the arrangement shown, as one example, an optimal position during back sleeping is one of spinal alignment, such that the nose is in line with the sternum while the head is fully supported. Furthermore, the optimal position places the ears over the shoulders and the head slightly extended to allow for optimal breathing and chest expansion. System 10 provides for this back sleeping position, other sleeping positions, and maintains these positions and ergonomics throughout the course of sleep, among other features, components, benefits, and functionalities.

System 10 also provides a neck arch 100. Neck arch 100, in the arrangement shown, as one example, shows a particular size. However, this size is variable and/or can be varied depending on the design and pillow provided. The neck arch 100, shown, provides for a shallow and short arch. This prevents pushing of the chin to the chest as is found in the present state of the art, further preventing poor breathing and/or chest compression, and/or strain on the spine and other ligaments and nerves.

Similarly, and in the arrangement shown, as one example, system 10 includes a left concavity and/or a first concavity formed of a tall and wide oval shape which supports the face and/or head during side sleeping. Similarly, and in the arrangement shown, as one example, system 10 includes a right concavity and/or a third concavity formed of a tall and wide oval shape which supports the face and/or head during side sleeping. Said another way, the concavity allows the face and head to cradle into the concave depression so the head sits perpendicular to the body, providing support and alignment.

Furthermore, and in the arrangement shown, as one example, system 10 provides additional concavities for other portions of a user. In the arrangement shown, as one example, system 10 includes a first arch 80 arrangement on the side of the first concavity which provides a slot for a user's shoulder. Similarly, system 10 provides a second arch 90 arrangement on the side of the second concavity which provides a second slot for a user's shoulder sleeping on the alternate side. In this way, the first arch and/or second arch provide for the user and/or sleeper to have an expanded chest which improves upon the ergonomics of a user. Similarly, in this way, the first arch and/or second arch provide for the user and/or sleeper to have an expanded chest which improves upon breathing. Similarly, in this way, the first arch and/or second arch provide for the user and/or sleeper to have an expanded chest which improves upon avoiding rounded shoulders of a user or causing hunching. In one alternative embodiment, a third arch 100 with a third cradle 108 is included in the center of the core.

Additionally, and in the arrangement shown, as one example, an arch approach 120 is included. Arch approach is configured as a regressed area with a form to specifically contour to the shoulder and neck area of a user while providing support.

In the arrangement shown, as one example, system 10 includes an insert 60 and/or core 60. The insert is placed and/or located within the outer covering which contains a comfort fill such as down feathers, feathers, or the like. The insert 60 (to be further described herein) provides the user the ability to maintain cervical lordosis and avoid anterior head translation relative to the body. Said another way, existing pillows in the market cause the cranium, or base of the skull to be strained at undesirable angles in relation to the body. Existing pillows create a very large gap between the head and the mattress resulting in compression or chin to chest flexion of the spine. The present disclosure and insert 60 prevent this debilitation.

Said another way, and in the arrangement shown, as one example, system 10 provides for side sleeping of a user in which the face rests comfortably in the depressed oval concavity. In this way, the system provides for a neutral spine and prevents lateral compression to the head and/or shoulders of a user. By contrast, existing pillows in the state of the art cause too much height to be expressed under the head and above the head which pushes the head away from the mattress and/or surface of sleep and/or rest.

Said another way, system 10 provides for optimal side sleeping. In the arrangement shown, as one example, system 10 causes the nose of a user to be in line with the sternum of a user. Furthermore, and in the arrangement shown, system 10 causes the ears to be perpendicular to a user's shoulders when side sleeping. Furthermore, and in the arrangement shown, as one example, system 10 causes the neck to be straight when side sleeping. This provides spine alignment and prevents strain.

Said another way, system 10 provides for improved and optimal side sleeping through the left and right concavities because the cradles of the left and right concavities are tall and optimized to cradle the entire head and face. The positioning of the left and right concavities provide only a short distance from the pillow base to the opening of the concavity, such that, the flexion of ear to shoulder is prevented. This further increases comfort, causes alignment, and provides for decreased strain and the like. In this way, system 10 provides for improved and/or optimized sleeping position.

In the arrangement shown, as one example, system 10 includes a plurality of concavities 18, an outer cover 20, an inner cover 40, a core or insert 60, a first arch 80, a second arch 90, and a neck arch 100, among other components, features, and functionality.

Furthermore, system 10, in one arrangement and in one example as shown herein can be approached by a user from either of the opposing sides. In this way, and in this embodiment, system 10 includes at least two neck arches. Said another way, system 10 is flippable in two directions or planes, as one example of how the system can be changed or used in alternative methods, so it can be used on the opposite sides. In this way, four arches for the neck in back sleeping are included, six concavities are included in this example, and four side arches for the neck during side sleeping and four shoulder spaces.

System 10 is not limited to this number of arches, concavities, and/or spaces. This is one example of one embodiment. Alternative examples include, but are not limited to one arch, two arches, three arches, four arches, five arches, six arches, seven arches, eight arches, nine arches, ten arches, or more. Similarly, alternative examples include, but are not limited to one concavity, two concavities, three concavities, four concavities, five concavities, six concavities, seven concavities, eight concavities, nine concavities, ten concavities, or more. Similarly, alternative embodiments include, but are not limited to one space, two spaces, three spaces, four spaces, five spaces, six spaces, seven spaces, eight spaces, nine spaces, ten spaces, or more.

### User (or Plurality Thereof)

In the arrangement shown, as one example, system 10 includes a user. User may be any user interacting with or utilizing the system 10. This may include viewing, controlling, analyzing, manipulating, and/or interacting with system 10. User is not limited to a single user but may be a plurality of users.

### Outer Cover

In the arrangement shown, as one example, system 10 includes an outer cover 20 (also referred to as “cover”). Cover 20 is formed of any suitable size, shape, and design, and is configured to provide a comfortable outer layer for a user to engage directly with. In the arrangement shown, as one example, outer cover 20 extends a length from a first end 22 to a second end 23 between opposing sides 24. Cover 20 also includes a top 26 and a bottom 28. Cover 20 also includes a hollow interior 30 (into which the core 60 is placed or already placed and/or changed out of). Cover 20 also includes an outer surface made of a soft and/or desirable fabric and/or material. Cover 20 also includes piping and/or stitching. Cover 20 may also include a layer of fill.

Furthermore, the outer cover is also configured to provide an aesthetically appealing device. Existing pillows which serve orthopedic purposes are irregular in shape, distorted in color and/or don't appear inviting and soft. The outer cover, as one example, provides comfort and an inviting aesthetic appeal for comfort and for decoration. The outer cover also causes the pillow to appear as a more typical pillow rather than an orthopedic pillow.

### Inner Cover

In the arrangement shown, as one example, system 10 may also include an inner cover 40 (also referred to as “second cover”, or “core cover”). Core cover 40 is formed of any suitable size, shape, and design, and is configured to provide a cover and/or secondary cover of the core 60 for added protection and the like.

Not all systems include an first inner cover 40. First inner cover 40 is only required in some embodiments. Inner cover 40 extends a length from a first end 42 to a second end 43 between opposing sides 44—having a top 46 and a bottom 48. Inner cover 40 may also have a hollow interior 50 and an outer surface 51. Inner cover 40 may also include a piping and/or stitching, as wall as a fill.

### Second Inner Cover

In the arrangement shown, as one example, system 10 may also include a second inner cover 140 (also referred to as “second cover”, or “core cover”). Second core cover 140 is formed of any suitable size, shape, and design, and is configured to provide a cover and/or secondary cover of the core 60 for added protection and the like.

Not all systems include a second inner cover 140. Second inner cover 140 is only required in some embodiments. Second inner cover 140 extends a length from a first end 142 to a second end 143 between opposing sides 144-having a top 146 and a bottom 148. Second inner cover 140 may also have a hollow interior 150 and an outer surface 151. Second inner cover 140 may also include a piping and/or stitching, as wall as a fill.

### Core

In the arrangement shown, as one example, system 10 includes a core 60 (also referred to as “inner core”, “insert”, “main body”, “latex contoured pillow” or simply “piece”). Insert 60 is formed of any suitable size, shape, and design, and is configured to provide the ergonomic benefits, support, and comfort of system 10 as described herein.

In the arrangement shown, as one example, system 10 extends a length from a first end 62 to a second end 63 between opposing sides 64. Core 60 also includes a top 66 and a bottom 68. Core includes a first indent and/or first concavity and/or left concavity 70 having a first arch 80 with a first cradle 88, wherein the first cradle and second cradle support the neck and shoulder area of a user. Insert 60 also includes a second indent and/or second concavity and/or head concavity 71 having a second arch 90 and a second cradle 98. Insert 60 also includes a third indent and/or third concavity 73 having a third arch 100 and a third cradle 108.

### Cell Structure

In the arrangement shown, as one example, the core 60 includes a cell structure 73. Cell structure 73 is formed of any suitable size, shape, and design, and is configured to provide support and comfort. In the arrangement shown, as one example, the cell structure 73 is formed of hexagonal cells interwoven with one another three-dimensionally so as to provide stability, movement, and support.

### First Nose and Second Nose

In the arrangement shown, as one example, core 60 includes a first nose 74 and a second nose 75. First nose 74 and second nose 75 are formed of any suitable size, shape, and design and are configured to help the core maintain its shape and support features near the outer edges of the core, via u-shaped outer edge features which are formed of a material more rigid than the core. Said another way, first nose 74 and second nose 75 are configured to maintain the appropriate curvature for various body types while maintaining shape and durability of the core while providing flexibility for various body types.

### Arch Approach

Additionally, and in the arrangement shown, as one example, an arch approach 110 is included. Arch approach is configured as a regressed area with a form to specifically contour to the shoulder and neck area of a user while providing support.

### Second Arch Approach

In the arrangement shown, as one example, a second arch approach 112 is included. Arch approach is configured as a regressed area with a form to specifically contour to the shoulder and neck area of a user while providing support.

### Access Port

In the arrangement shown, as one example, the outer cover includes an access port 120. Access port 120 is configured to provide access to the interior so the inner layer and the core can be inserted and/or removed. This aids in cleaning the outer cover and the like. First Access port 120, in the arrangement shown, as one example, is a zipper. However, other access ports are hereby contemplated for use such as an opening, a hook and loop, buttons, and the like.

### Second Access Port

In the arrangement shown, as one example, the inner cover includes a second access port 130. Second access port 130 is configured to provide access to the interior so the core can be inserted and/or removed. This aids in cleaning the outer cover and the like. Second Access port 130, in the arrangement shown, as one example, is an opening. However, other access ports are hereby contemplated for use such as a zipper, a hook and loop, buttons, and the like.

### Third Access Port

In the arrangement shown, as one example, the second inner cover includes a third access port 160. Third access port 160 is configured to provide access to the interior so the core can be inserted and/or removed. This aids in cleaning the outer cover and the like. Third Access port 160, in the arrangement shown, as one example, is an opening. However, other access ports are hereby contemplated for use such as a zipper, a hook and loop, buttons, and the like.

### First and Second Sidearm

The core also includes a first and second sidearm. The first and second sidearm provide supports for arms and/or other uses for stabilization and comfort.

### Sensor System

In the arrangement shown, as one example, system 10 has a plurality of sensors. The plurality of sensors are configured to detect the position of a user. More specifically, and without limitation, the plurality of sensors are configured to detect the pressure and presence of a users head, a users neck, and a users shoulders. The plurality of sensors, along with the computing components and computer processing system can determine the pressure of the head, the shoulders, and the neck and determine if a different concavity should be utilized to either raise or lower the head position relative to the sleeping surface. In this way, the pressure can be reduced, the curvature of the shoulder reduced, the spine put into a more neutral and ergonomic position. This provides for improved neck and spine alignment, improved shoulder health, and improved breathing. All of these combine for an improved night of sleep, providing improved duration, comfort, and postures.

The sensor system may be located in the core via a plurality of sensors located throughout various parts. Sensors located in the cover are also hereby contemplated for use, such as sensors for heart rate monitoring, sleep detection, and the like. The sensor system is configured to communicate via bluetooth with a smart device to provide information and input for computing and processing. The system may recommend the user change positions from front, side, to back. Similarly, the sensor system may recommend the user change to a different concavity as a selection from the plurality of concavities.

In addition to the above identified features, options, controls, and components, system 10 may also include other features and functionalities, among other options, controls, and components.

It will be appreciated by those skilled in the art that other various modifications could be made to the system, process, and method of use without parting from the spirit and scope of this disclosure. All such modifications and changes fall within the scope of the claims and are intended to be covered thereby.

1. An ergonomic pillow system, comprising:
an outer cover;
the outer cover extending a length from a first end to a second end;
the outer cover having a top and a bottom;
the outer cover having a hollow interior and an outer surface;
a core;
the core extending a length from a first end to a second end;
the core having a top and a bottom;
the core having a first concavity;
the core having a second concavity;
the core having a first arch;
the first arch having a first cradle;
the core having a second arch;
the second arch having a second cradle;
wherein the first concavity is on the top of the core;
the first concavity having a concavity forming a first cradle;
wherein the second concavity is on the top of the core;
the second concavity having a concavity forming a second cradle.

2. The system of claim 1, further comprising:
wherein the first cradle supports the neck and shoulder area of a user.

3. The system of claim 1, further comprising:
the core having a third concavity.

4. The system of claim 1, further comprising:
the outer cover including a fill;
an inner cover;
the inner cover extending a length from a first end to a second end;
the inner cover having a top and a bottom;
the inner cover having a hollow interior and an outer surface;
the inner cover having an access port.

5. The system of claim 1, further comprising:
a second inner cover;
the second inner cover extending a length from a first end to a second end;
the second inner cover having opposing sides;
the second inner cover having a top and a bottom;
the second inner cover having a hollow interior and an outer surface;
the second inner cover having an access port.

6. The system of claim 1, further comprising:
the core having a third concavity;
the core having a fourth concavity;
wherein a depth of the first concavity, a depth of the second concavity, a depth of the third concavity, and a depth of the fourth concavity are all different depths.

7. The system of claim 1, further comprising:
the core having a third concavity;
wherein the third concavity is on the top of the core;
the third concavity having a third arch forming a third cradle;
wherein a depth of the first concavity, a depth of the second concavity, a depth of the third concavity are all different depths;
wherein the third concavity decreases in depth moving outward from a center of the third concavity to an outer ring of the third concavity.

8. The system of claim 1, further comprising:
the core having a third concavity;
wherein the third concavity is more shallow than the first concavity;
the core having a fourth concavity;
wherein the third concavity is on the bottom of the core;
wherein the fourth concavity is on the bottom of the core.

9. The system of claim 1, further comprising:
the core having a cell structure;
wherein the cell structure forms the interior material of the core;
the core having a sensor system; the sensor system having a plurality of sensors;
wherein the sensor system is configured to detect the presence of a sleeper;
wherein the sensor system is configured to detect the pressure of a head of the sleeper relative to the core; wherein the sensor system is configured to detect the pressure of the shoulders of the sleeper relative to the core; wherein the sensor system is configured to detect the pressure of the neck of the sleeper relative to the core;
wherein the sensor system relays a set of inputs to a smart device, the smart device provides computing and returns an output of a positional change recommendation if the positional change recommendation is required; wherein the positional change recommendation might be a change in the position of the sleeper and a change of concavity selection.

10. The system of claim 1, further comprising:
the core having a first nose;
wherein the first nose is configured to maintain the shape of the core by providing rigidity through outer edge u-shaped stabilization features;
the core having a second nose;
wherein the first nose is configured to maintain the shape of the core by providing rigidity through outer edge u-shaped stabilization features.

11. The system of claim 1, further comprising:
the core having an arch support;
the arch support located at the superior and inferior borders;
the arch support leads from a plurality of concavities to an edge of the opposing sides of the core;
the core having an arch approach;
the arch approach formed of a recessed area in the lower center of the core;
wherein the arch approach is configured to form a contour which accepts the shoulder and neck area of a user; providing support to a user engaging with the system in a position selected from the following: providing support to a user laying on a back position; providing support to a user laying on a stomach position; providing support to a user laying on a side position;
wherein the arch approach provides space for a shoulder of a user which allows for appropriate expansion of a chest of the user during breathing, providing improved breathing during sleep;
wherein the arch approach prevents the shoulder of the user from rounding or curving forward, providing an improved ergonomic position during sleep.

12. The system of claim 1, further comprising:
the core having an arch approach;
the arch approach formed of a recessed area in the lower center of the core;
wherein the arch approach is configured to form a contour which accepts the shoulder and neck area of a user; providing support to a user engaging with the system in a position selected from the following: providing support to a user laying on a back position; providing support to a user laying on a stomach position; providing support to a user laying on a side position;
the core having a second arch approach;
the second arch approach formed of a recessed area in the upper center of the core;
wherein the second arch approach is configured to form a contour which accepts the shoulder and neck area of a user; providing support to a user engaging with the system in a position selected from the following: providing support to a user laying on a back position; providing support to a user laying on a stomach position; providing support to a user laying on a side position.

13. The system of claim 1, further comprising:
the core having a first sidearm;
wherein the first sidearm is configured to provide a resting position for the arm of a user;
the core having a second sidearm;
wherein the second sidearm is configured to provide a resting position for the arm of the user;
wherein when the user is resting in a side sleeping position, the core provides support for a head of the user in one of a plurality of concavities selected from the first concavity or the second concavity; wherein the core places a spine of the user in a neutral position, the neutral position being a straight line when viewed from the side, preventing strain on any portion of the spine through varying depths of the plurality of concavities; wherein the support of the plurality of concavities prevents lateral compression to the head and shoulders of the user, preventing the head of the user being pushed away from a sleep surface, providing for improved breathing by allowing for expansion of the chest since the shoulders of the user are not being curved and pressed into a torso of the user.

14. The system of claim 1, further comprising:
wherein the first concavity provides support for the head of a user;
wherein the first concavity prevents the head of the user from changing positions while sleeping due the concave shape of the first concavity;
wherein the first arch provides support to the neck of the user;
wherein the neck of the user engages with the first arch while the head of the user is in the first concavity;
wherein the first concavity is configured to maintain cervical lordosis and prevent anterior head translation relative to the body of the user, providing a position such that the head of the user is not pushed forward by supporting the head of the user while also providing for a base of the head of the user to rest supported and close to a mattress.

15. A pillow insert apparatus, comprising:
a core;
the core extending a length from a first end to a second end;
the core having opposing sides;
the cover having a top and a bottom;
the cover having a first indent;
the core having a second indent;
the core having a third indent;
the core having a fourth indent;
wherein the first indent is on the top of the core;
wherein the second indent is on the top of the core.

16. The system of claim 15, further comprising:
wherein the first indent provides support for the head of a user;
wherein the first indent prevents the head of the user from changing positions while sleeping due the concave shape of the first indent;
wherein the first arch provides support to the neck of the user;
wherein the neck of the user engages with the first arch while the head of the user is in the first indent.

17. The system of claim 15, further comprising:
wherein the cell structure forms the interior material of the core; the core having a first nose;
wherein the first nose is configured to maintain the shape of the core by providing rigidity through outer edge u-shaped stabilization features;
the core having a second nose;
wherein the first nose is configured to maintain the shape of the core by providing rigidity through outer edge u-shaped stabilization features;
the core having an arch approach;
the arch approach formed of a recessed area in the lower center of the core;
wherein the arch approach is configured to form a contour which accepts the shoulder and neck area of a user; providing support to a user laying on a back position; providing support to a user laying on a stomach position; providing support to a user laying on a side position;
the core having a second arch approach;
the second arch approach formed of a recessed area in the upper center of the core;
wherein the second arch approach is configured to form a contour which accepts the shoulder and neck area of a user; providing support to a user laying on a back position; providing support to a user laying on a stomach position; providing support to a user laying on a side position;
the core having a first sidearm;
wherein the first sidearm is configured to provide a resting position for the arm of a user;
the core having a second sidearm;
wherein the second sidearm is configured to provide a resting position for the arm of a user.

18. The system of claim 15, further comprising:
wherein the third indent is on the top of the core;
the third indent having a concave shape forming a third cradle.

19. The system of claim 15, further comprising:
wherein the third indent is on the bottom of the core;
the third indent having a concave shape forming a third cradle.

20. An ergonomic pillow system, comprising:
an outer cover;
the outer cover extending a length from a first end to a second end;
the outer cover having opposing sides;
the outer cover having a top and a bottom;
the outer cover having a hollow interior and an outer surface;
the outer cover having an access port;
an inner cover;
the inner cover extending a length from a first end to a second end;
the inner cover having opposing sides;
the inner cover having a top and a bottom;
the inner cover having a hollow interior and an outer surface;
the inner cover having an access port;
a second inner cover;
the second inner cover extending a length from a first end to a second end;
the second inner cover having opposing sides;
the second inner cover having a top and a bottom;
the second inner cover having a hollow interior and an outer surface;
the second inner cover having an access port a core;
the core extending a length from a first end to a second end;
the core having opposing sides;
the cover having a top and a bottom;
the cover having a first indent;
the core having a second indent;
the core having a third indent;
the core having a fourth indent;
wherein the first indent is on the top of the core;
the first indent having a concave shape forming a first cradle;
wherein the second indent is on the top of the core;
the second indent having a concave shape forming a second cradle;
wherein the fourth indent is on the bottom of the core;
the third indent having a third a concave shape a third cradle;
the core having a cell structure;
wherein the cell structure forms the interior material of the core;
the core having a first nose;
wherein the first nose is configured to maintain the shape of the core by providing rigidity through outer edge u-shaped stabilization features;
the core having a second nose;
wherein the first nose is configured to maintain the shape of the core by providing rigidity through outer edge u-shaped stabilization features;
the core having an arch approach;
the arch approach formed of a recessed area in the lower center of the core;
wherein the arch approach is configured to form a contour which accepts the shoulder and neck area of a user; providing support to a user laying on a back position; providing support to a user laying on a stomach position; providing support to a user laying on a side position;
the core having a second arch approach;
the second arch approach formed of a recessed area in the upper center of the core;
wherein the second arch approach is configured to form a contour which accepts the shoulder and neck area of a user; providing support to a user laying on a back position; providing support to a user laying on a stomach position; providing support to a user laying on a side position;
the core having a first sidearm;
wherein the first sidearm is configured to provide a resting position for the arm of a user;
the core having a second sidearm;
wherein the second sidearm is configured to provide a resting position for the arm of a user;
wherein the core is configured to provide improved sleep to a user; the core improving the ergonomics of a user by supporting the head and neck areas of a user; the first indent and second indent providing variation in depth for various user preferences;
wherein the third indent is more shallow than the first indent and the second indent;
wherein the first arch and the second arch are configured to support the neck area of the user;
wherein the first cradle and the second cradle are configured to support the neck area of the user while also preventing a user from rolling from side to side along the length of the core.
